(19) United States
(12) Patent Application Publication
(10) Pub. No.: US 2025/0264428 A1
(43) Pub. Date: Aug. 21, 2025

(54) GAS SENSOR

(71) Applicant: TDK Corporation, Tokyo (JP)

(72) Inventor: Kei TANABE, Tokyo (JP)

(21) Appl. No.: 19/197,153

(22) Filed: May 2, 2025

Related U.S. Application Data
(63) Continuation of application No. PCT/JP2022/044418, filed on Dec. 1, 2022.

(51) Int. Cl. G01N 27/14; G01N 33/00

(52) U.S. Cl. CPC G01N 27/14; G01N 33/0027

(57) ABSTRACT

Disclosed herein is a gas sensor that includes: first and second thermistors connected in series; first and second heaters configured to heat the first and second thermistors, respectively; and a control circuit configured to control the first and second heaters and apply a power supply to the first and second thermistors In a first period, the control circuit is configured to: start heating the first and second heaters; start applying the power supply to the first and second thermistors after a start of heating the first and second heaters; and generate an output signal indicating a concentration of a gas to be measured based on a detection signal appearing at a connection point between the first and second thermistors in a state where the first and second heaters are heated and the power supply is applied to the first and second thermistors.

### BACKGROUND OF THE ART

### Field of the Art

The present disclosure relates to a gas sensor.

### Description of Related Art

International Publication WO 2020/031517 discloses a gas sensor that detects the concentration of a gas to be measured based on the level of a detection signal appearing at the connection point between two thermistors connected in series between a power supply and a ground. The gas sensor described in International Publication WO 2020/031517 heats a thermistor constituting a detection element and a thermistor constituting a reference element to 150° C. and 300° C., respectively, to acquire a detection signal.

In the gas sensor described in International Publication WO 2020/031517, power consumption occurs during current flow through the two series-connected thermistors.

### SUMMARY

A gas sensor according to the present disclosure includes: first and second thermistors connected in series; first and second heaters for heating the first and second thermistors, respectively; and a control circuit configured to control the first and second heaters and apply a power supply to the first and second thermistors. In a first period, the control circuit is configured to: start heating the first and second heaters; start applying the power supply to the first and second thermistors after a start of heating the first and second heaters; and generate an output signal indicating a concentration of a gas to be measured based on a detection signal appearing at a connection point between the first and second thermistors in a state where the first and second heaters are heated and the power supply is applied to the first and second thermistors.

### BRIEF DESCRIPTION OF THE DRAWINGS

The above features and advantages of the present disclosure will be more apparent from the following description of some embodiments taken in conjunction with the accompanying drawings, in which:

FIG. 1 is a circuit diagram illustrating the configuration of a gas sensor 1 according to a first embodiment of the technology described herein;

FIG. 2 is a timing chart for explaining a first operation example of the gas sensor 1;

FIG. 3 is a graph illustrating the temperature characteristics of the thermistors Rd1 and Rd2;

FIG. 4 is a graph illustrating the relation between the temperature of the thermistors Rd1, Rd2 and their sensitivity to CO₂ gas;

FIG. 5 is a graph for explaining a temporal change of the heating time of the thermistors Rd1 and Rd2 in period T1;

FIG. 6 is a timing chart for explaining a second operation example of the gas sensor 1;

FIG. 7 is a circuit diagram illustrating the configuration of the gas sensor 2 according to the second embodiment of the technology described herein;

FIGS. 8A and 8B are circuit diagrams showing examples of the resistance measurement circuit 11; and

FIG. 9 is a timing chart for explaining the operation of the gas sensor 2.

### DETAILED DESCRIPTION OF THE EMBODIMENTS

The present disclosure describes a technology for reducing power consumption in a gas sensor having two series-connected thermistors.

Some embodiments of the present disclosure will be explained below in detail with reference the accompanying drawings.

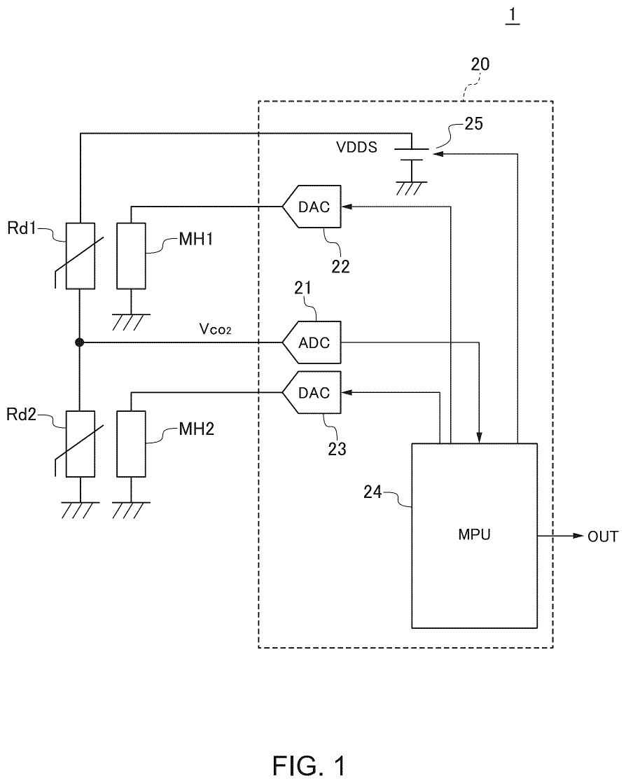

FIG. 1 is a circuit diagram illustrating the configuration of a gas sensor 1 according to a first embodiment of the technology described herein.

As illustrated in FIG. 1, the gas sensor 1 according to the first embodiment includes thermistors Rd1 and Rd2, heater resistors MH1 and MH2 for heating the thermistors Rd1 and Rd2, respectively, and a control circuit 20 for controlling the heater resistors MH1 and MH2. Although not particularly limited, the gas sensor 1 according to the present embodiment is a thermal conduction type gas sensor for detecting the concentration of CO₂ gas in the atmosphere.

The thermistors Rd1 and Rd2 are detection elements made of a material having a negative temperature coefficient of resistance, such as a composite metal oxide, amorphous silicon, polysilicon, or germanium. Both the thermistors Rd1 and Rd2 detect the concentration of CO₂ gas, but have different operating temperatures, as described below. Here, the thermistor Rd1 constitutes a detection element, and the thermistor Rd2 constitutes a reference element. The thermistors Rd1 and Rd2 are connected in series between a power supply 25 for supplying a power supply potential VDDS and a ground, and a detection signal Vco2 appearing at the connection point between the thermistors Rd1 and Rd2 is supplied to the control circuit 20.

The control circuit 20 includes an AD converter (ADC) 21, DA converters (DAC) 22 and 23, an MPU 24, and the power supply 25. The AD converter 21 AD-converts the detection signal Vco2 appearing at the connection point between the thermistors Rd1 and Rd2 and supplies an obtained digital value to the MPU 24. The MPU 24 generates an output signal OUT indicating the concentration of CO₂ gas based on the AD-converted detection signal. The DA converters 22 and 23 DA-convert the digital value supplied from the MPU 24 to apply predetermined voltages to the heater resistors MH1 and MH2, respectively. In other words, the heating temperatures of the heater resistors MH1 and MH2 are controlled by the MPU 24.

The following describes the operation of the gas sensor 1 according to the present embodiment.

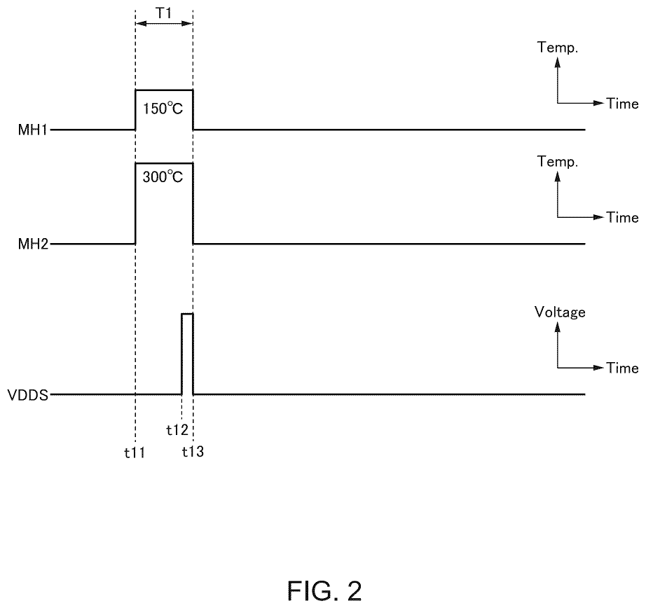

FIG. 2 is a timing chart for explaining a first operation example of the gas sensor 1 according to the present embodiment.

In the first operation example illustrated in FIG. 2, gas measurement operation is performed in period T1. In the gas measurement operation, the heater resistors MH1 and MH2 are heated to 150° C. and 300° C., respectively, under the control of the MPU 24. The heater resistor MH1 and thermistor Rd1 are disposed in close proximity to each other and thus, the temperature of the heater resistor MH1 can be regarded as substantially the esame as the temperature of the thermistor Rd1. Similarly, the heater resistor MH2 and thermistor Rd2 are disposed in close proximity to each other and thus, the temperature of the heater resistor MH2 can be regarded as substantially the same as the temperature of the thermistor Rd2.

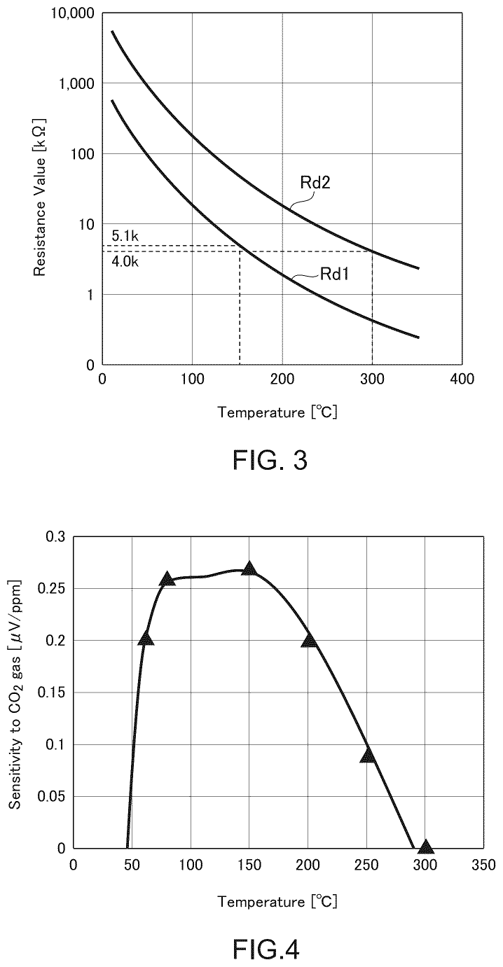

As illustrated in FIG. 3, the temperature characteristics of the thermistors Rd1 and Rd2 are mutually different and designed such that the resistance value of the thermistor Rd1 heated to 150° C. and the resistance value of the thermistor Rd2 heated to 300° C. are close to each other. In the example illustrated in FIG. 3, the resistance value of the thermistor Rd1 heated to 150° C. is 5.1 kΩ, and the resistance value of the thermistor Rd2 heated to 300° C. is 4.0 kΩ. The resistance value of the thermistor Rd1 heated to 150° C. and the resistance value of the thermistor Rd2 heated to 300° C. may be approximately the same.

FIG. 4 is a graph illustrating the relation between the temperature of the thermistors Rd1, Rd2 and their sensitivity to CO₂ gas. As can be seen from the graph of FIG. 4, the sensitivity of the thermistors Rd1 and Rd2 to CO₂ gas varies significantly depending on the temperature, and the sensitivity of the thermistors Rd1, Rd2 to CO₂ gas is almost zero in the temperature range below 40° C. or above 300° C. The sensitivity of the thermistors Rd1, Rd2 to CO₂ gas is maximum at about 150° C.

Thus, when CO₂ gas is present in the measurement atmosphere with the thermistor Rd1 as the detection element heated to 150° C., the heat dissipation characteristics of the thermistor Rd1 change according to the concentration. Such a change appears as a change in the resistance value of the thermistor Rd1. On the other hand, even when CO₂ gas is present in the measurement atmosphere with the thermistor Rd2 as the reference element heated to 300° C., the heat dissipation characteristics of the thermistor Rd2 hardly change according to the concentration. Therefore, the change in the resistance value of the thermistor Rd2 heated to 300° C. due to the concentration of CO₂ gas is sufficiently smaller than the change in the resistance value of the thermistor Rd1 heated to 150° C. due to the concentration of CO₂ gas. There is no problem if the resistance value of the thermistor Rd2 heated to 300° C. due to the concentration of CO₂ gas hardly changes.

As a result, the level of the detection signal Vco2 appearing at the connection point between the thermistors Rd1 and Rd2 changes according to the concentration of CO₂ gas in the measurement atmosphere. The detection signal Vco2 is supplied to the MPU 24 through the AD converter 21, and the MPU 24 generates the output signal OUT indicating the concentration of CO₂ gas based on the supplied detection signal Vco2.

During the gas measurement operation performed in period T1, the heating start timing of the heater resistors MH1 and MH2 is time t11, the power supply application start timing to the thermistors Rd1 and Rd2 by the power supply 25 is time t12, and the heating end timing of the heater resistors MH1 and MH2 and the power supply application end timing to the thermistors Rd1 and Rd2 by the power supply 25 are time t13. Thus, the heater resistors MH1 and MH2 are heated during a period from time t11 to time t13, and the thermistors Rd1 and Rd2 are applied a power supply during a period from time t12 to time t13. The power supply application time to the thermistors Rd1 and Rd2 is sufficiently shorter than the heating time of the heater resistors MH1 and MH2 and is about one-tenth thereof, for example. As described above, in the present embodiment, the power supply application timing to the thermistors Rd1 and Rd2 is made later than the heating start timing of the heater resistors MH1 and MH2.

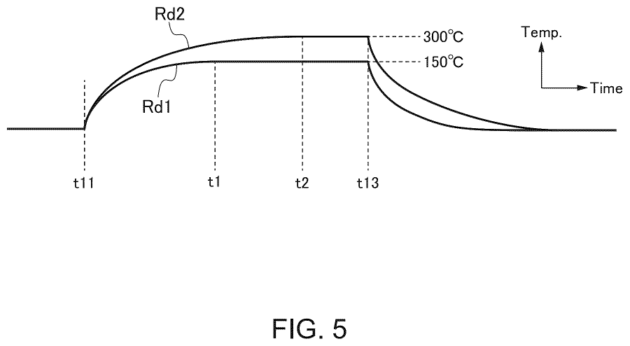

FIG. 5 is a graph for explaining a temporal change of the heating time of the thermistors Rd1 and Rd2 in period T1.

As illustrated in FIG. 5, when the heating of the heater resistors MH1 and MH2 is started at time t11, the temperatures of the thermistors Rd1 and Rd2 rise. However, a predetermined time is required until the temperature of the thermistor Rd1 reaches a target value of 150° C., and a predetermined time is required until the temperature of the thermistor Rd2 reaches a target value of 300° C. In the example of FIG. 5, the temperature of the thermistor Rd1 reaches the target value of 150° C. at time t1, and the temperature of the thermistor Rd2 reaches the target value of 300° C. at time t2. That is, even after the start of heating of the heater resistors MH1 and MH2, the concentration of CO₂ gas cannot be properly measured by the thermistors Rd1 and Rd2 until the time t2 is reached. It is only after the time t2 that the concentration of CO₂ gas can be properly measured. Therefore, even when a power supply is applied to the thermistors Rd1 and Rd2 before time t2, the detection signal Vco2 obtained before time t2 cannot be sampled. Considering this, in the present embodiment, the power supply application start timing (time t12) to the thermistors Rd1 and Rd2 is delayed, whereby unnecessary power consumption is reduced.

Although time t12 at which the power supply application to the thermistors Rd1 and Rd2 is started is not particularly limited provided that it is after time t11 and before t13; however, by setting time t12 to time t2 when the concentration of CO₂ gas can be properly measured or a time close to time t2, it is possible to make a time during which a power supply is applied to the thermistors Rd1 and Rd2 shortest. Then, the detection signal obtained between time t12 and t13 is sampled by the MPU 24, and the output signal OUT indicating the concentration of CO₂ gas is calculated based on the sampled detection signal.

The start and end of the power supply application to the thermistors Rd1 and Rd2 may be performed by the MPU 24 in the following manner: controlling the operation of the power supply 25 that generates and terminates the power supply potential VDDS; or turning ON/OFF a switch provided at the output end of the power supply 25. Further, the power supply 25 need not necessarily be a constant voltage source but may be a constant current source. Furthermore, the heating start timing of the heater resistor MH1 and that of the heater resistor MH2 need not necessarily be the same as each other, but the heating of the heater resistor MH2 to be heated to a higher temperature may be started earlier than the heater resistor MH1.

Further, the timing of time t12 is not particularly limited and may be timing when a predetermined period passes from time t11 at which heating of the heater resistors MH1 and MH2 is started. Thus, the relation between the time t11 and time t12 is fixed, facilitating control by the MPU 24. Alternatively, a period from time t11 at which the heating of the heater resistors MH1 and MH2 is started to time t12 at which the power supply application to the thermistors Rd1 and Rd2 is started may be made variable.

As described above, in the first operation example, the power supply application to the thermistors Rd1 and Rd2 is started after the start of heating of the heater resistors MH1 and MH2, so that power consumption by the thermistors Rd1 and Rd2 can be reduced. In addition, the thermistors Rd1 and Rd2 are reduced also in self-heating amount and are thus suppressed in aging. Note that it is not necessary to make the heating end timing of the heater resistors MH1 and MH2 and power supply end timing to the thermistors Rd1 and Rd2 completely coincide with each other; however, by making them coincide with each other, the occurrence of unnecessary power consumption can be prevented.

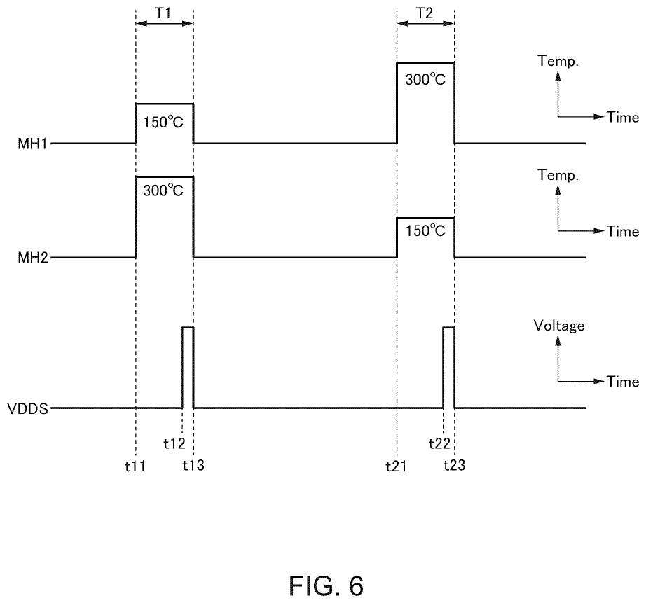

FIG. 6 is a timing chart for explaining a second operation example of the gas sensor 1 according to the present embodiment.

In the second operation example illustrated in FIG. 6, the gas measurement operation is performed in period T1, and dummy heating operation is performed in period T2. The gas measurement operation and dummy heating operation are alternately performed. The gas measurement operation performed in period T1 is the same as that in the first operation example, so overlapping description will be omitted.

As illustrated in FIG. 6, during the dummy heating operation performed in period T2, the heater resistors MH1 and MH2 are heated to 300° C. and 150° C., respectively, under the control of the MPU 24. This eliminates the thermal history difference between the thermistors Rd1 and Rd2 generated during the gas measurement operation performed in period T1. To eliminate the thermal history difference more accurately, the lengths of periods T1 and T2 should be made to coincide with each other. Further, the detection signal Vco2 is not sampled in period T2, so that the power supply application to the thermistors Rd1 and Rd2 may be stopped completely. This contributes to further reduction in power consumption. Alternatively, as illustrated in FIG. 6, a configuration may be employed in which the heating of the heater resistors MH1 and MH2 is started at time t21, the power supply application to the thermistors Rd1 and RD2 is started at time t22, and the heating of the heater resistors MH1 and MH2 and the power supply application to the thermistors Rd1 and Rd2 by the power supply 25 are ended at time t23. As a result, the thermal history associated with the self-heating of the thermistors Rd1 and Rd2 in period T1 and the thermal history associated with the self-heating of the thermistors Rd1 and Rd2 in period T2 coincide with each other, making it possible to further reduce the thermal history difference between the thermistors Rd1 and Rd2. In this case, the power consumption and power supply application time of the thermistors Rd1 and Rd2 in period T1 and those in period T2 may be made to coincide with each other.

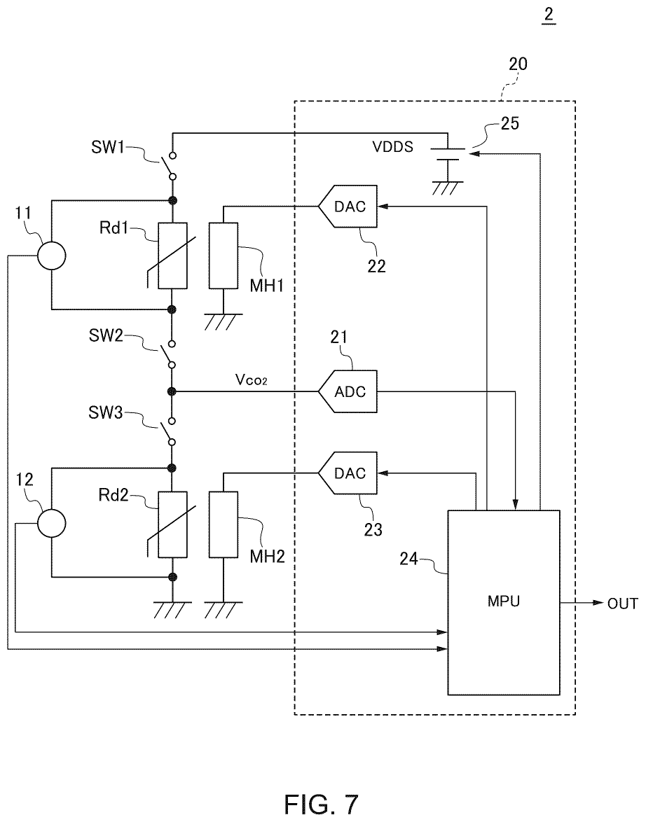

FIG. 7 is a circuit diagram illustrating the configuration of the gas sensor 2 according to the second embodiment of the technology described herein.

As illustrated in FIG. 7, the gas sensor 2 according to the second embodiment differs from the gas sensor 1 according to the first embodiment in that it additionally includes resistance measurement circuits 11, 12 and switches SW1 to SW3. Other basic configurations are the same as those of the gas sensor 1 according to the first embodiment, so the same reference numerals are given to the same elements, and overlapping description will be omitted.

The switch SW1 is connected between the power supply 25 for supplying the power supply potential VDDS and the thermistor Rd1. The switches SW2 and SW3 are connected between the thermistors Rd1 and Rd2. As a result, when the switches SW1 to SW3 are turned ON, the thermistors Rd1 and Rd2 are connected in series between the power supply 25 and the ground. The potential appearing between the switches SW2 and SW3 in this state, i.e., the detection signal Vco2 appearing at the connection point between the thermistors Rd1 and Rd2 is supplied to the control circuit 20. On the other hand, when the switches SW1 to SW3 are turned OFF, the series connection of the thermistors Rd1 and Rd2 is released, and the thermistors Rd1 and Rd2 are separated from each other. The resistance measurement circuits 11 and 12 measure the resistance values of the thermistors Rd1 and Rd2, respectively, in a state where the switches SW1 to SW3 are turned OFF.

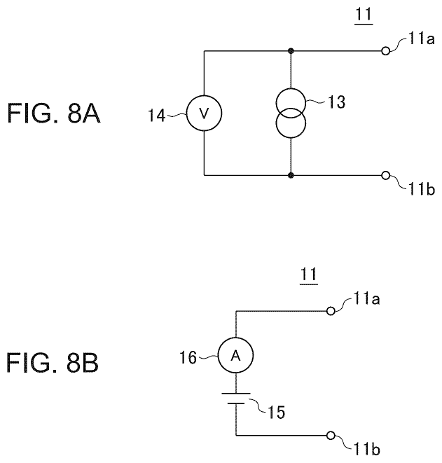

As illustrated in FIG. 8A, the resistance measurement circuit 11 may have a configuration in which a constant current source 13 and a voltmeter 14 are connected in parallel between one end 11a and the other end 11b. With this configuration, when a constant current is made to flow from the constant current source 13 to the thermistor Rd1 in a state where the thermistor Rd1 is connected between the one end 11a and the other end 11b, a voltage to be generated between the one end 11a and the other end 11b is determined by the resistance value of the thermistor Rd1. This voltage is measured with the voltmeter 14 and supplied to the control circuit 20. This allows the control circuit 20 to acquire the directly-measured resistance value of the thermistor Rd1.

Alternatively, as illustrated in FIG. 8B, the resistance measurement circuit 11 may have a configuration in which a constant voltage source 15 and an ammeter 16 are connected in series between the one end 11a and the other end 11b. With this a configuration, when predetermined voltage is applied from the constant voltage source 15 to the thermistor Rd1 in a state where the thermistor Rd1 is connected between the one end 11a and the other end 11b, a current flowing between the one end 11a and the other end 11b is determined by the resistance value of the thermistor Rd1. This current is measured with the ammeter 16 and supplied to the control circuit 20. This allows the control circuit 20 to acquire the directly-measured resistance value of the thermistor Rd1.

The same applies to the configuration of the resistance measurement circuit 12, and the control circuit 20 can acquire the directly-measured resistance value of the thermistor Rd2.

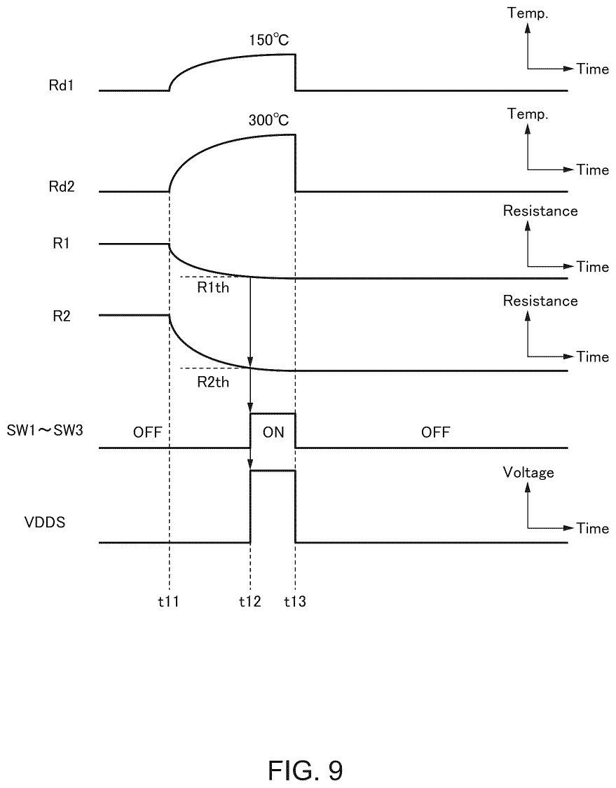

FIG. 9 is a timing chart for explaining the operation of the gas sensor 2 according to the present embodiment.

As illustrated in FIG. 9, in the present embodiment, the switches SW1 to SW3 are turned OFF at time t11, and thereby the MPU 24 monitors resistance values R1 and R2 of the respective thermistors Rd1 and Rd2 measured by the resistance measurement circuits 11 and 12, respectively. Then, in response to the resistance value R1 of the thermistor Rd1 reaching a predetermined value R1th and the resistance value R2 of the thermistor Rd2 reaching a predetermined value R2th, the switches SW1 to SW3 are turned ON, and the power supply application to the thermistors Rd1 and Rd2 is started. The predetermined value R1th is a threshold value that the resistance value R1 exceeds when the thermistor Rd1 is heated to about 150° C. irrespective of the concentration of CO₂ gas contained in the atmosphere. Similarly, the predetermined value R2th is a threshold value that the resistance value R2 exceeds when the thermistor Rd2 is heated to about 150° C. irrespective of the concentration of CO₂ gas contained in the atmosphere. This allows the power supply application to the thermistors Rd1 and Rd2 to be started in a state where the concentration of CO₂ gas can be properly measured by the thermistors Rd1 and Rd2.

However, it is not essential to monitor both the resistance values R1 and R2 of the thermistors Rd1 and Rd2, but it is only necessary to monitor one of the resistance values R1 and R2. That is, in response to one of the resistance values R1 and R2 reaching a predetermined value, the switches SW1 to SW3 are turned ON, and the power supply application to the thermistors Rd1 and Rd2 is started. In this case, the resistance value of the thermistor Rd2 heated to a higher temperature may be monitored in period T1 during which the gas measurement operation is performed, and the resistance value of the thermistor Rd1 heated to a higher temperature may be monitored in period T2 during which the dummy heating operation is performed.

While some embodiments of the technology according to the present disclosure have been described, the technology according to the present disclosure is not limited to the above embodiments, and various modifications may be made within the scope of the present disclosure, and all such modifications are included in the technology according to the present disclosure.

For example, although the measurement target gas is CO₂ gas in the above embodiments, the present invention is not limited to this. Further, the sensor part used in the present invention need not necessarily be a thermal conduction type sensor, but may be a sensor of other types such as a catalytic combustion type. As an example, when the measurement target gas is CO gas, a catalytic combustion type sensor part can be used.

The technology according to the present disclosure includes the following configuration examples, but not limited thereto.

A gas sensor according to the present disclosure includes: first and second thermistors connected in series; first and second heaters for heating the first and second thermistors, respectively; and a control circuit configured to control the first and second heaters and apply a power supply to the first and second thermistors. In a first period, the control circuit is configured to: start heating the first and second heaters; start applying the power supply to the first and second thermistors after a start of heating the first and second heaters; and generate an output signal indicating a concentration of a gas to be measured based on a detection signal appearing at a connection point between the first and second thermistors in a state where the first and second heaters are heated and the power supply is applied to the first and second thermistors. This can reduce power consumed by the first and second thermistors.

In the above gas sensor, in the first period, the control circuit may be configured to start applying the power supply to the first and second thermistors after an elapse of a predetermined period from the start of heating the first and second heaters. This facilitates control by the control circuit.

In the above gas sensor, in the first period, the control circuit may be configured to control a period from the start of heating the first and second heaters to a start of applying the power supply to the first and second thermistors. This allows the power supply application to the first and second thermistors to be started at an optimum timing.

In the above gas sensor, in the first period, the control circuit may be configured to start applying the power supply to the first and second thermistors in response to a resistance value of the first or second thermistor reaching a predetermined value after the start of heating the first and second heaters. This allows the power supply application to the first and second thermistors to be started in a state where the concentration of a gas to be measured can be properly measured.

In the above gas sensor, in the first period, the control circuit may be configured to make a heating end timing of the first and second heaters and an applying end timing of the power supply to the first and second thermistors coincide with each other. This facilitates control and can prevent the occurrence of unnecessary power consumption.

In the above gas sensor, the control circuit may be configured to heat the second heater to a higher temperature than a heating temperature of the first heater in the first period, and the control circuit may be configured to heat the first heater to a higher temperature than a heating temperature of the second heater in a second period. This can reduce a difference in thermal history between the first and second thermistors.

In the above gas sensor, in the second period, the control circuit may be configured to start applying the power supply to the first and second thermistors after a start of heating the first and second heaters. This reduces also a difference in thermal history due to the self-heating of the first and second thermistors.

In the above gas sensor, the control circuit may be configured to: make a heating temperature and a heating time of the first heater in the first period and a heating temperature and a heating time of the second heater in the second period coincide with each other; make a heating temperature and a heating time of the second heater in the first period and a heating temperature and a heating time of the first heater in the second period coincide with each other; make a power consumption of the first and second thermistors in the first period and a power consumption of the first and second thermistors in the second period coincide with each other; and make an application time of the power supply to the first and second thermistors in the first period and an application time of the power supply to the first and second thermistors in the second period coincide with each other. This can reduce the thermal history difference between the first and second thermistors more accurately.

What is claimed is:

1. A gas sensor comprising:
first and second thermistors connected in series;
first and second heaters configured to heat the first and second thermistors, respectively; and
a control circuit configured to control the first and second heaters and apply a power supply to the first and second thermistors,
wherein, in a first period, the control circuit is configured to:
start heating the first and second heaters;
start applying the power supply to the first and second thermistors after a start of heating the first and second heaters; and
generate an output signal indicating a concentration of a gas to be measured based on a detection signal appearing at a connection point between the first and second thermistors in a state where the first and second heaters are heated and the power supply is applied to the first and second thermistors.

2. The gas sensor as claimed in claim 1, wherein, in the first period, the control circuit is configured to start applying the power supply to the first and second thermistors after an elapse of a predetermined period from the start of heating the first and second heaters.

3. The gas sensor as claimed in claim 1, wherein, in the first period, the control circuit is configured to control a period from the start of heating the first and second heaters to a start of applying the power supply to the first and second thermistors.

4. The gas sensor as claimed in claim 3, wherein, in the first period, the control circuit is configured to start applying the power supply to the first and second thermistors in response to a resistance value of the first or second thermistor reaching a predetermined value after the start of heating the first and second heaters.

5. The gas sensor as claimed in claim 1, wherein, in the first period, the control circuit is configured to make a heating end timing of the first and second heaters and an applying end timing of the power supply to the first and second thermistors coincide with each other.

6. The gas sensor as claimed in claim 1,
wherein the control circuit is configured to heat the second heater to a higher temperature than a heating temperature of the first heater in the first period, and
wherein the control circuit is configured to heat the first heater to a higher temperature than a heating temperature of the second heater in a second period.

7. The gas sensor as claimed in claim 6, wherein, in the second period, the control circuit is configured to start applying the power supply to the first and second thermistors after a start of heating the first and second heaters.

8. The gas sensor as claimed in claim 7, wherein the control circuit is configured to:
make a heating temperature and a heating time of the first heater in the first period and a heating temperature and a heating time of the second heater in the second period coincide with each other;
make a heating temperature and a heating time of the second heater in the first period and a heating temperature and a heating time of the first heater in the second period coincide with each other;
make a power consumption of the first and second thermistors in the first period and a power consumption of the first and second thermistors in the second period coincide with each other; and
make an application time of the power supply to the first and second thermistors in the first period and an application time of the power supply to the first and second thermistors in the second period coincide with each other.
